(19) United States
(12) Patent Application Publication
(10) Pub. No.: US 2007/0282272 A1
(43) Pub. Date: Dec. 6, 2007

(54) Device for guiding medical tubing

(76) Inventors: Chad D. Bannon, Venice, CA (US); Anton Posniak, Marina Del Rey, CA (US)

Correspondence Address: GREENBERG TRAURIG LLP (LA), 2450 COLORADO AVENUE, SUITE 400E, INTELLECTUAL PROPERTY DEPARTMENT, SANTA MONICA, CA, 90404, US

(21) Appl. No.: 11/443,812

(22) Filed: May 30, 2006

(51) Int. Cl. A61M 5/32

(52) U.S. Cl. 604/174

(57) ABSTRACT

A device for guiding and/or organizing medical tubing for use in a hospital, especially associated with a rail of a hospital bed, an intravenous pole, portion of a wheelchair, or a sheet material associated with the bed or patient. In one embodiment, the device comprises a base and a cylindrical lateral loop or enclosure, which is a tubing guide. The loop guide is affixed with a base. The device allows free movement of the tube within the enclosure and thus greater patient mobility.

### BACKGROUND

The disclosure relates to medical tubing. More specifically, it relates to a device for guiding and/or organizing medical tubing for use in a medical facility. Even more specifically, it relates to a tubing organizer or guide for use in a hospital, especially associated with a rail of a hospital bed, an intravenous pole, portion of a wheelchair, or a sheet material associated with the bed or patient.

### SUMMARY

The disclosure is a device for guiding and/or organizing medical tubing that allows medical tubing to be held in a convenient location proximate to a patient while allowing the tubing to move freely within the device, thus, giving the patient a greater range of movement.

As used herein, the term “medical tubing” or “tubing” encompasses any tubing or conduit consisting of a long object (usually cylindrical) used to hold and/or conduct objects, liquids, gases, electrical current, cabling, or transmit data appropriate for use in the medical field. For example, medical tubing can comprise an intravenous tube.

In one embodiment, the tube is held in a generally cylindrical lateral loop or enclosure, which is a tubing guide. The tubing guide is affixed to a base. In one embodiment, the cylindrical lateral loop or enclosure, although affixed to the base, is movable relative to the base. The base, in one form, is a clip. In another form, the base is a flexible material such as a strap. In one embodiment, the base includes a substantially flat base portion to which the tubing guide is attached. While the preferred embodiment depicts a generally circular tubing guide, the tubing guide may be any other shape that would efficiently guide the medical tubing. For example, the tubing guide could be elliptical, triangular, rectangular or any other shape that would retain the tubing but allow it to move easily in the general manner described herein.

The base could be a clip or a strap or the like, so that the base can engage a rail, handrail, bar, or strut of a hospital bed, whether the rail, handrail, bar or strut is horizontal, vertical or oriented in another direction. In another embodiment, the base (whether in clip form or strap form) can engage vertically or horizontally with any type of pole, including a moveable pole, associated with medical tubing, such as an intravenous pole or stand. Further, the clip can be easily transferred from a relatively fixed structure such as a bed, to a mobile unit such as an intravenous stand and/or a wheelchair. Thus, in each embodiment the entire unit is held in relatively close proximity to the patient to allow the patient's medical tubing to be held near the patient while still allowing the medical tubing to move freely within the cylindrical, or other shaped, lateral loop or enclosure, giving the patient a greater range of movement. In one embodiment, the cylindrical, or other shaped, lateral loop or enclosure is capable of rotating or moving relative to the base to allow for a further range of movement. In an alternate embodiment, the tube-guiding portion can be removed from the base, if desired, to facilitate turning or relocating the patient.

In one form, the base and the tubing guide, which can also be referred to as a loop, cylindrical loop or enclosure, are formed of two different elements and/or materials, which are fixedly mounted with one another. The base can be of a metal or plastic material and the enclosure member of a different metal or plastic material. In another embodiment, where the base is in a form of a strap, the base can be of a plastic, fabric, nylon, latex, spandex, neoprene, or any other material capable of being shaped as a strap and wrapped or deformed around a surface such as a bedrail, pole, or stand. In another embodiment, these materials are the same or similar.

In another embodiment, the disclosure provides for a device for guiding medical tubing, or a tubing organizer, wherein the tubing guide portion is within a hinged apparatus that can be releasably opened and closed to facilitate attachment of the apparatus to a sheet or blanket or other device or structure near the patient.

In another embodiment, the base portion is formed of one or two parts and has an aperture for receiving and guiding a tubing.

The disclosure provides a device for guiding medical tubing where the base portion of the device is easily attachable and detachable to the side rail of a hospital bed, an intravenous pole, wheelchair or other stationary or moveable structure near a patient.

The device, and some of the alternative embodiments, is described in the following specification and drawings.

### DRAWINGS

FIG. 1A is a perspective view of a device for guiding medical tubing according to a first embodiment.

FIG. 1B is a side view of a device for guiding medical tubing according to the first embodiment.

FIG. 1C is a side view of a device for guiding medical tubing according to the first embodiment.

FIG. 1D is a side view of a guide loop, cylindrical loop, or enclosure portion of a device for guiding medical tubing according to an embodiment.

FIG. 1E is a side view of a guide loop, cylindrical loop, or enclosure portion of a device for guiding medical tubing according to an embodiment.

FIG. 1F is a perspective view of a device for guiding medical tubing according to a second embodiment.

FIG. 2 is an environmental perspective view of the medical implement and tubing organizer in a second embodiment.

FIG. 3 is an environmental perspective view of the medical implement and tubing organizer in a third embodiment.

FIG. 4 is an environmental perspective view of the medical implement and tubing organizer in a fourth embodiment.

FIGS. 5A, 5B, 5C, 5D, and 5E are cross sectional view of different cross sectional frame, rail, pole, or stand structures.

### DESCRIPTION

A device for guiding and/or organizing medical tubing that guides medical tubing in a convenient location proximate to a patient while allowing the tubing to move freely within the device, which gives the patient a greater range of movement, is described.

In one embodiment, the device comprises a base portion for securing the device to a supporting surface and an enclosure for receiving at least one medical tube. Further, the device comprises a releasable and reusable interface provided for repeatedly securing the enclosure member about at least one tube. As used herein the term “supporting surface” may comprise a rail or other portion of a hospital bed, an intravenous pole, portion of a wheelchair, or a sheet material associated with the bed or patient. The supporting surface may be any other device or structure near the patient. Thus, the base portion of the device secures the device to the supporting surface.

In an alternate embodiment, the base portion further includes a rest element for supporting at least a portion of an arm, wrist, or hand of a patient.

In another embodiment, the base portion may comprise a strap with an interface. For example, the interface can include a hook and loop type fastener, and in some embodiments, the base portion and enclosure are formed of a tape. The base portion includes a hook and loop type fastener to facilitate anchoring of the base to a support surface. For example, the base portion includes an adjustable engagement comprising a strap having hook and loop type engaging elements for attachment to bed frames having differing cross-sections. Thus, the base is for securing to a bed rail, and the bed rail includes selectively a cross section being at least one of boxed, curved, box/rectangle with curved corners, circular, and wherein the enclosure member is for guiding at least one of the cords or wires or tubes in a medical setting.

In one embodiment, the base portion may comprise a clip. For example, the base portion includes an adjustable engagement element for attaching the base portion to a supporting surface. As another example, the base portion includes a clip for attachment to bed frames having different cross-sections and different shapes.

In one embodiment, regardless of whether the base portion is in the form of a clip or strap, the base portion can lie in a first plane and the enclosure is upright relative to the base. The enclosure member allows for relatively free movement of the tubing within the enclosure member and thus allows for greater patient mobility. In one form of use, there is a single enclosure member only secured with the base.

In one embodiment, the tube-guiding portion of the enclosure is angularly rotatable about an axis through the point of joinder between the base and enclosure. Because the enclosure is capable of rotating about the base, there is a greater range of movement for the tubing and thus, the patient. In one embodiment, the enclosure is permanently attached to the base. In another embodiment, the enclosure member is removable from and reattachable to the base.

In another alternate embodiment, the device for guiding medical tubing for use with a patient comprises a base portion having a supporting surface for supporting and guiding the tubing. Further, the device comprises a guide as part of the base portion for receiving at least one medical tube, such as an intravenous injection tube. A releasable and reusable guide is available for repeatedly engaging and disengaging the medical tubing. In this embodiment, the base portion includes at least a portion of an aperture to guide the medical tubing. The device in this embodiment includes a tubing guide within two elements that are urged into engagement by a biasing element. A common example of a basic structure would be a clothespin or alligator clip. In this embodiment, the tubing guide is part of, or integral with, the base portion. The base includes a portion for attachment to a sheet material, such as a bed sheet, blanket or the patient's clothing. The base portion includes a clip for attachment to the sheet. As such, the base portion can include at least two components having mating faces wherein the tube-guiding aperture is located, at least partly, in each of the mating faces. In another form, the base portion includes at least two components, the components having mating faces wherein the tube-guiding aperture is located in a single one of the mating faces. In all cases, the tube-guiding aperture is sized so the medical implement or tubing inserted into the guide aperture is free to move in the aperture, the aperture being designed to inhibit snagging.

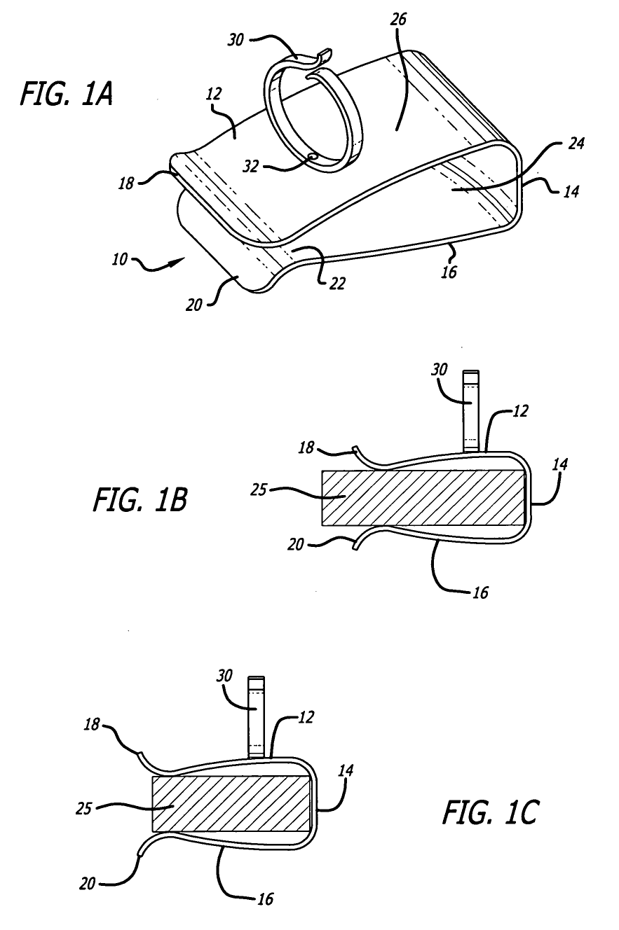

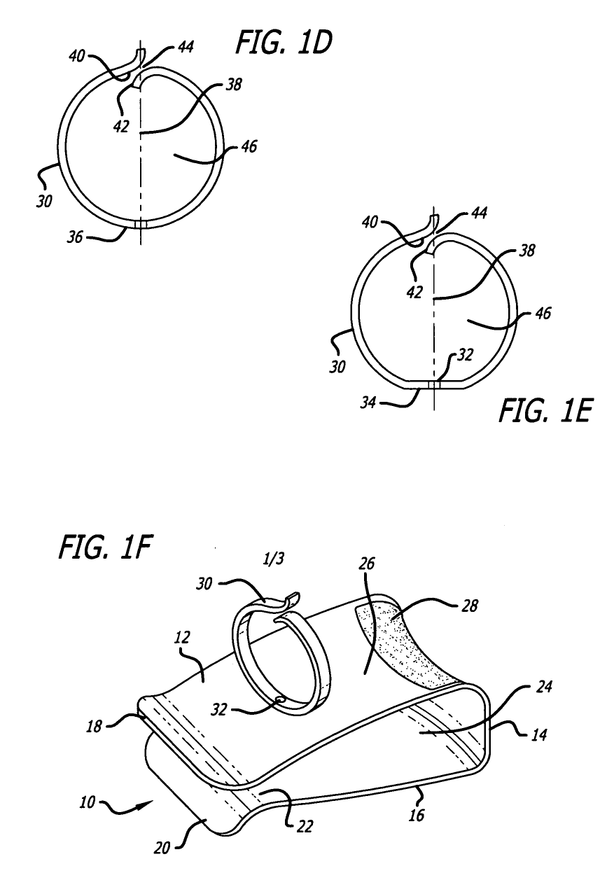

FIG. 1A shows one embodiment of the present disclosure. In FIG. 1A, the device comprises a base 10, which is in the form of a clip having an upper element 12, a downwardly depending backward element 14, and a base section 16. The free end 18 of element 12 and free end 20 of element 16 are curved away from each other to form the lips of a mouth area 22, which is narrower than the space cavity 24 between the ends 18 and 20. Inside of the mouth 22 and back wall 14, cavity 24 is disposed for receiving a supporting surface such as a rail of a hospital bed. This is the element for supporting the clip or base 10 with respect to the bed, intravenous pole, and/or other supporting surface. FIGS. 1B and 1C depict examples of the clip or base 10 engaging with a supporting surface such as a bed rail 25. As can be seen in FIGS. 1B and 1C, the clip can engage with the bed rail (or any other type of supporting surface) and enclose the bed rail as in FIG. 1C or partially enclose the bed rail as shown in FIG. 1B.

On the top plate 12 of the base 10 there is located an enclosure 30, which is in the form of a loop. The loop may be circular, elliptical, triangular, rectangular, or another shape consistent with the operation of the device. The loop 30 can be fixed by a bolt or pivot 32 to the top plate 12 in an appropriate manner. The enclosure 30 can have a flat surface 34 as shown in FIG. 1E or be rounded as indicated by 36 in FIG. 1D. By having the round surface 36 as shown in FIG. 1D, the enclosure may be more easily relatively rotatable about an axis 38, which passes substantially vertically through the enclosure 30 so that the enclosure 30 can rotate around 360° as necessary. Of course, the terms “vertical,” “vertically,” “horizontal,” and “horizontally” are relative to the drawings, whereas the device itself can be used in various orientations relative to the horizon. This rotation allows the tubing, and thus the patient, a greater range of movement. Appropriate different stops, detents or clips can be provided between the interface of the enclosure 30 and the top plate 12 so that different locking positions or friction positions are provided so that the enclosure 30 can be secured in different relative positions with respect to the base 10 as necessary.

The free ends of the enclosure 30 define an interface between ends 40 and 42, which can provide a gap 44 through which a tube or implement can be inserted. In some cases, the gap 44 can be more closed or more open according to different configurations. The ends of the interface elements 40 and 42 are flared remotely from each other to facilitate the ingress and egress of the tubes or implements from the bore 46, which is inside of the enclosure.

The base and/or enclosure may be made of metal or of some plastic material. These materials can be relatively different and have different degrees of flexibility and elasticity. A prerequisite amount of elasticity and flexibility maintains interface elements 40 and 42 at the proper distance from each other to insure that the tubes and/or implements remain within the bore while allowing the interface elements 40 and 42 to be spread open sufficiently to allow the medical device or tubing to be inserted into the bore 46. Likewise, flexibility in the panels 12, 14, and 16 of the clip can provide for easier affixing of the base portion of the clip to a bed rail or other device.

An alternate embodiment is shown in FIG. 1F. The embodiment in FIG. 1F is similar to the embodiment depicted in FIG. 1A, except that the top face 26 of the device is curved and scalloped and may be placed on the arm, hand, or wrist of a patient. A padded or friction surface 28 is provided around the bottom portion of the back wall 14 and overlapping onto the top face of the wall 12 in order to facilitate gripping.

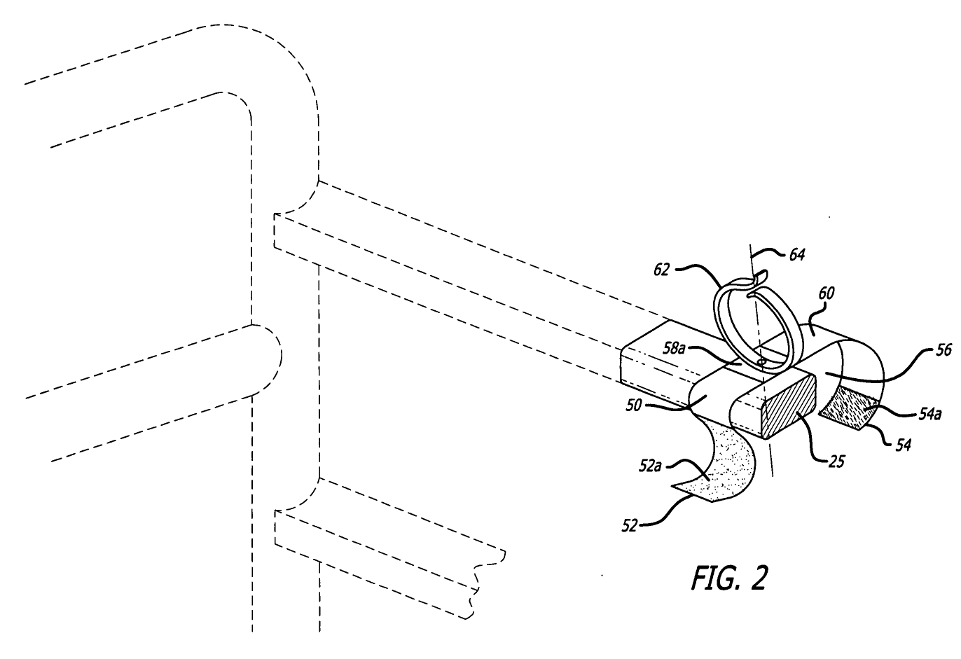

As shown in FIG. 2, there is an embodiment wherein the base is formed at least in part by a flexible strap 50. The strap may be made of a plastic, fabric, nylon, latex, spandex, neoprene, or any other material capable of being shaped as a strap and wrapped or deformed around a supporting surface such as a bed rail, pole, or stand. As depicted in FIG. 2, the strap 50 has free ends 52 and 54, which may include hook and loop-type formations 52a and 54a to facilitate enclosure in a locking relationship. The bed rail would be located inside the space 56 as defined by the closed strap 50. A similar hook and loop formation 58a can be provided on the top face 60 of the strap 50. A mating hook and loop formation 58b on the base of the enclosure member 62 can be provided for securing the enclosure 62 with the base strap portion 50. In other embodiments, the enclosure 62 may be secured to the top face of the strap 50 using a pin or similar type of fastener. With this relationship, the enclosure member 62 can also be rotated about an axis 64 so that the relative orientations of the strap 50 and the enclosure member 62 can be changed.

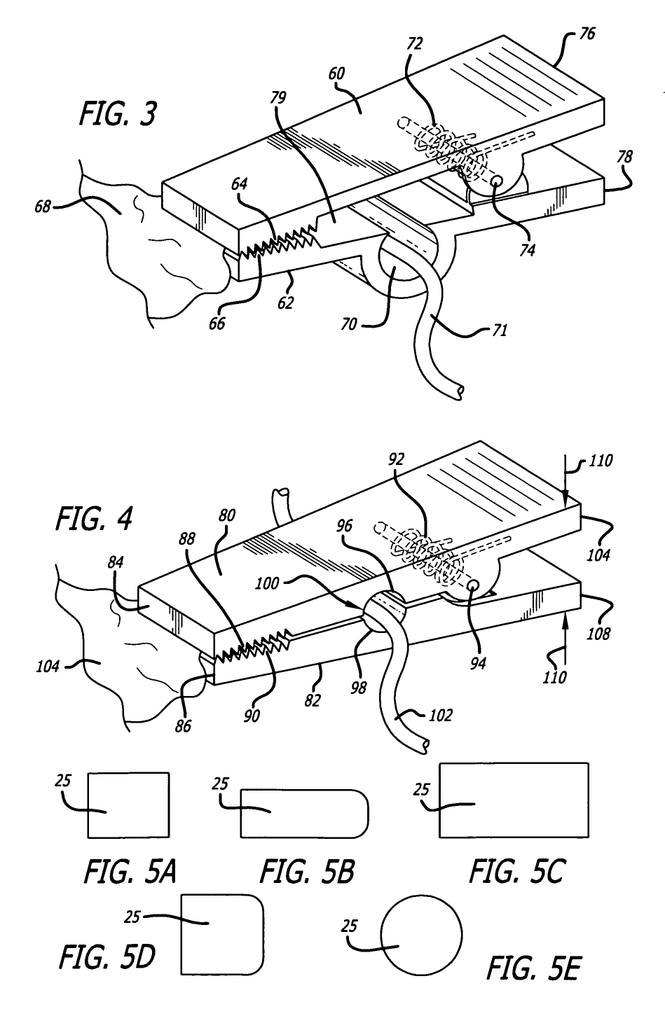

Another embodiment is illustrated in FIG. 3. As depicted in FIG. 3, the device is a clothespin type style device. The base includes an upper member 60 and lower member 62 respectively with teeth 64 and 66, which are at the open ends of the two clip members 60 and 62. The teeth 64 and 66 can inter-engage as necessary.

There can also be different sheet materials 68 for engaging between the teeth 62 and 64. The sheet material can be a bed sheet, blanket, or clothing of a patient. In the lower element 62 of the base there is a bore or tunnel 70 through which a medical tubing or implement can be located. In this configuration, the bore 70 is located essentially in only one of the elements of the base member. A spring 72 can be located around an axle 74, which biases the elements 60 and 62 towards each other so that the mouth is closed in a manner similar to the operation of a conventional clothespin. The ends 76 and 78 of the clips are urged together under finger action by an operator causing the mouth at the free ends 62 and 64 to open and permit passage of tubing 71 into the cavity 79 and in turn into the bore 70. The tubing 71 could pass between the teeth 64 and 66. In an alternative embodiment, tubing 71 may be able to pass in and through the bore 70 without opening the mouth of the clip.

FIG. 4 depicts an embodiment, which is similar to the clothespin style clamp depicted in FIG. 3, except the shape of the bore, or tunnel differs. As can be seen in FIG. 4, the base is formed by an upper element 80 and a lower element 82 with leading ends 84 and 86 in a normal position of closure. Teeth 88 and 90 engage to effect closure. A spring element 92 inside the elements 80 and 82 urge the ends 84 and 86 together. A suitable pivot axis 94 is provided about which the elements 80 and 82 can open and close.

Two half portions 96 and 98 of a bore 100 are in opposite mating relationships to form substantially the bore 100 through which a tube 102 can pass as required. A sheet material 104 is engaged between the open ends 84 and 86 to secure thereby the device near the patient as necessary.

In one embodiment, a mouth of the clip can be opened and the tube 102 passed into the bore. In other cases with the mouth closed, the tube 102 can pass in and out of the bore. The diameter of the bore and tube are sized to facilitate different operational configurations that are appropriate to the situation. In different cases, the bore 100 can be smoothed or rounded at the edges to facilitate the smooth movement of the tube in and out of the bore as necessary.

The clip of FIG. 4 is operated by urging the ends 104 and 108 together by finger pressure as indicated by arrows 110. The release of the finger pressure allows the mouth members at 84 and 86 to move towards closure.

As disclosed in FIGS. 5A, 5B, 5C, 5D, and 5E, different configurations of bed rails or other supporting structures are possible, and the devices disclosed in this specification can be used or adapted to be used on the different cross sections illustrated in these figures, or for other cross sections. The cross sections 25 can be relatively square to rectangular to circular and can have square or rounded edges, etc. The bed rails may be side rails, vertical rails, or oblique rails in different configurations. The rails may have any thickness such as ¾″, ⅝″, ⅞″, 1″ or other size.

It is to be understood that the disclosed device is not limited to the embodiments described above, but encompasses any and all embodiments within the scope of the following claims.

Although certain illustrative embodiments and methods have been disclosed herein, it will be apparent form the foregoing disclosure to those skilled in the art that variations and modifications of such embodiments and methods may be made without departing from the true spirit and scope of the art disclosed. Many other examples of the art disclosed exist, each differing from others in matters of detail only. Accordingly, it is intended that the art disclosed shall be limited only to the extent required by the appended claims and the rules and principles of applicable law.

1. A device for guiding medical tubing for use with a patient comprising:
a base portion for supporting the device on a supporting surface;
an enclosure for receiving at least one of a medical tubing; and
a releasable and reusable interface for repeatedly holding the enclosure member about at least one of the tubing, wherein the base portion lies in a first plane and the enclosure is upright relative to the base.

2. The device for guiding medical tubing according to claim 1, wherein the interface includes a hook and loop type fastener.

3. The device for guiding medical tubing according to claim 1, wherein the interface includes a hook and loop type fastener, and the base portion and enclosure are formed of a tape, the base portion including a hook and loop type fastener to facilitate anchorage of the base to a support.

4. The device for guiding medical tubing according to claim 1, wherein the upright portion relationship of the enclosure is angularly rotatable about a relative right angular axis through the point of joinder between the base and enclosure.

5. The device for guiding medical tubing according to claim 1, wherein the enclosure is permanently attached to the base.

6. The device for guiding medical tubing according to claim 1, wherein the base portion includes an adjustable engagement element for attaching the base portion to said supporting surface.

7. The device for guiding medical tubing according to claim 1, wherein the supporting surface comprises a bed rail.

8. The device for guiding medical tubing according to claim 1, wherein the supporting surface comprises a mobile pole for supporting an intravenous line.

9. The device for guiding medical tubing according to claim 1, wherein the base portion includes an adjustable engagement comprising a strap having hook and loop type engaging elements for attachment to supporting surfaces having a different cross-section.

10. The device for guiding medical tubing according to claim 1, wherein there is a single enclosure member only secured with the base.

11. The device for guiding medical tubing according to claim 1, wherein the enclosure member allows for relatively free movement of the cords or wires or tubes within the enclosure member.

12. The device for guiding medical tubing according to claim 1, wherein the base is for securing to a bed rail, and wherein the bed rail includes selectively a cross section being at least one of square, curved, rectangular, rectangular with curved corners, circular, and wherein the enclosure member is for holding at least one of cords or wires or tubes in a medical setting; and wherein the enclosure member allows for relatively free movement of the cords or wires or tubes within the enclosure member.

13. A device for guiding medical tubing for use with a patient comprising:
a base portion for supporting the device on a supporting surface;
an enclosure as part of the base portion for receiving at least one of a medical tubing;
a releasable and reusable interface for repeatedly holding the enclosure member about at least one of the tubing; and
the base portion including at least a portion of an aperture for the medical implement or tubing.

12. The device for guiding medical tubing according to claim 11, wherein the interface includes two elements formed to be urged into engagement, and including a biasing element for urging the inter-engagement.

13. The device for guiding medical tubing according to claim 11, wherein the interface is part of the base portion.

14. The device for guiding medical tubing according to claim 11, wherein the base portion includes a portion for attachment to a sheet material, selectively a bed sheet, blanket, or patient clothing.

15. The device for guiding medical tubing according to claim 11, wherein the base portion includes a clip for attachment to the bed sheet.

16. The device for guiding medical tubing according to claim 11, wherein the base portion includes at least two components, the components having mating faces and wherein the aperture is for location at least partly in each of the mating faces.

17. The device for guiding medical tubing according to claim 11, wherein the base portion includes at least two components, the components having mating faces and wherein the aperture is for location in a single one of the mating faces.

18. The device for guiding medical tubing according to claim 16, wherein there is a single aperture for the enclosure member.

19. The device for guiding medical tubing according to claim 11, wherein the base portion includes at least two components, the components having mating faces, and wherein the aperture is sized for location of the medical implement or tubing whereby the medical implement or tubing is free to move in the aperture, the aperture being designed to inhibit friction and snag.
